(19) United States
(12) Patent Application Publication
(10) Pub. No.: US 2018/0249967 A1
(43) Pub. Date: Sep. 6, 2018

(54) DEVICES, SYSTEMS, AND ASSOCIATED METHODS FOR EVALUATING A POTENTIAL STROKE CONDITION IN A SUBJECT

(71) Applicant: Intel Corporation, Santa Clara, CA (US)

(72) Inventors: Dror Lederman, Qiryat-Gat (IL); Matan Touti, Jerusalem (IL); Ke Han, Shanghai (CN)

(73) Assignee: Intel Corporation, Santa Clara, CA (US)

(21) Appl. No.: 15/756,045

(22) PCT Filed: Sep. 25, 2015
(86) PCT No.: PCT/CN2015/090816

§ 371 Date: Feb. 27, 2018

(51) Int. Cl. A61B 5/00; A61B 5/11; G16H 50/30; A61B 5/0402

(52) U.S. Cl. CPC A61B 5/7282; A61B 5/1118; A61B 5/04014; A61B 5/0402; A61B 5/7246; G16H 50/30

(57) ABSTRACT

Devices (100), systems (200), and methods for automatically evaluating a potential stroke condition in a subject are disclosed and described.

### BACKGROUND

Cerebrovascular accident, or Stroke, is a condition that affects blood vessels supplying the brain with blood, and is a major cause of death and disability around the world. According to the World Health Organization (WHO), 15 million people worldwide suffer from stroke each year. Of these, 5 million die, and another 5 million are permanently disabled.

There are two major types of stroke: Hemorrhagic stroke and ischemic stroke. Hemorrhagic stroke accounts for about 13% of stroke cases, and results from a weakened vessel that ruptures and bleeds into the surrounding brain. The blood accumulates and compresses the surrounding brain tissue, causing serious brain damage that often results in death. Ischemic stroke results from an interruption of the supply of blood to brain cells caused by a blockage of one of the arteries in the brain. Affected brain cells can be deprived of oxygenated blood, and unless blood supply is restored, will begin to rapidly die. Due to the resulting cerebral hypoxia, affected neuronal regions can struggle or even fail to function. Depending on what those neuronal regions are, serious long-term disability or death can occur.

Early treatment of stroke is crucial, and can improve the prognosis and reduce neurological damage to the subject. One of the milestones of modern management of acute stroke is the administration of a thrombolytic (clot-busting medication) in order to unblock the affected arteries. It has been shown in international multi-center studies that patients who receive thrombolytic treatment often have improved clinical outcomes.

In recent years there has been significant progress in the treatment of stroke conditions, including the approval of several alternative thrombolytic drugs having enhanced safety. This progress can allow a longer time window between the onset of symptoms and latest safe time for treatment, the so-called ‘Time to Needle.’ During this time-window, subjects need to receive a neurological scan to rule out a hemorrhagic stroke or a tumor that would contraindicate the use of a thrombolytic.

### BRIEF DESCRIPTION OF THE DRAWINGS

FIG. 1 is a schematic view of a device for evaluating a potential stroke condition in a subject in accordance with an invention embodiment;

FIG. 2 is a schematic view of a system for evaluating a potential stroke condition in a subject in accordance with an invention embodiment;

FIG. 3 is an illustration of a method for evaluating a potential stroke condition in a subject in accordance with an invention embodiment;

FIG. 4a is a graphical representation of data in accordance with an invention embodiment;

FIG. 4b is a graphical representation of data in accordance with an invention embodiment;

FIG. 5 is an illustration of a machine learning algorithm in accordance with an invention embodiment;

FIG. 6a is a graphical representation of data in accordance with an invention embodiment;

FIG. 6b is a graphical representation of data in accordance with an invention embodiment;

FIG. 6c is a graphical representation of data in accordance with an invention embodiment;

FIG. 6d is a graphical representation of data in accordance with an invention embodiment;

FIG. 7a is a graphical representation of data in accordance with an invention embodiment;

FIG. 7b is a graphical representation of data in accordance with an invention embodiment; and

FIG. 8 is a graphical representation of data in accordance with an invention embodiment.

### DESCRIPTION OF EMBODIMENTS

Although the following detailed description contains many specifics for the purpose of illustration, a person of ordinary skill in the art will appreciate that many variations and alterations to the following details can be made and are considered to be included herein.

Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term in the specification, like “comprising” or “including,” it is understood that direct support should be afforded also to “consisting essentially of” language as well as “consisting of” language as if stated explicitly and vice versa.

“The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein.

As used herein, “enhanced,” “improved,” “performance-enhanced,” “upgraded,” and the like, when used in connection with the description of a device or process, refers to a characteristic of the device or process that provides measurably better form or function as compared to previously known devices or processes. This applies both to the form and function of individual components in a device or process, as well as to such devices or processes as a whole.

As used herein, the term “stroke condition” refers to a situation whereby a subject has experienced a stroke event.

As used herein, the term “stroke indicator” refers to a physiological, morphological, behavioral, psychological, or other sign or symptom that is merely suggestive of a stroke event in a subject.

As used herein, the term “feature indicator” refers to a measurable, noticeable, or otherwise detectable feature or characteristic of a stroke indicator. In some cases, a stroke indicator can be a feature indicator.

As used herein, the term “bilateral asymmetry” refers to a measurable, noticeable, or otherwise detectable asymmetry between two sides or two parts of a subject that is suggestive of a stroke event.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. However, it is to be understood that even when the term “about” is used in the present specification in connection with a specific numerical value, that support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

An initial overview of technology embodiments is provided below and specific technology embodiments are then described in further detail. This initial summary is intended to aid readers in understanding the technology more quickly, but is not intended to identify key or essential technological features, nor is it intended to limit the scope of the claimed subject matter.

Early detection of a stroke event is highly important, and has been shown to be highly correlated with improved prognosis. Unfortunately, in many cases a subject may feel a weakness or other symptoms, but might not be aware that such is a first sign of a developing stroke. Many subjects either cannot recall when symptoms started or are unable to communicate due to the neurological effects of the stroke. There is an unmet need, therefore, for a diagnostic-assist device that can alert of possible stroke.

Additionally, while there are several clinical tests which have been shown to be reliable in detecting stroke, including Cincinnati Prehospital Stroke Scale (CPSS), National Institute of Health Stroke Scale (NIHSS), and the Los Angeles Prehospital Stroke Screen (LAPSS), these tests require experience and high skills and can for the most part be performed only by professionals, i.e., neurologists. As such, a fully automatic device capable of detecting a stroke condition as it occurs is desirable.

Invention embodiments relate to devices, systems, and methods for the early detection of a stroke or a potential stroke condition in a subject. In one general example, sensors are positioned to gather data from the subject relating to the subject's physiology, bilateral motor performance, bilateral strength performance, body position, body symmetry including limb and facial symmetry, speech characteristics, or any other stroke-related characteristic or combination of characteristics. The data is then sent to a device or system where it can be integrated, analyzed, and/or monitored to provide an early warning of the stroke or potential stroke condition. Such a system can acquire, either continuously or intermittently, sensor data from a subject that is further analyzed using a smart asymmetry assessment or other algorithm.

In some cases, incoming data can be analyzed for a potential stroke indication, such as, for example, a bilateral asymmetry that may be associated with a stroke. Once detected, the device or system can perform further processing to test for a stroke or a potential stroke condition. Thus, in one example a device can analyze and monitor sensor data to determine the level of bilateral asymmetry in the subject. Sensor data patterns suggestive of a potential stroke condition can be further analyzed, in some cases along with additional data from the same and/or different sensors, to assess the likelihood of an actual stroke or stroke condition.

In one example, data from sensors measuring arm movement, position, rotation, etc. is sent to an analytic system, where such data is analyzed to determine the level of bilateral asymmetry presented by the subject. At some threshold level, the bilateral asymmetry can be suggestive of a potential stroke, and therefore further action can be triggered and/or implemented by the system. Various other sensor data, such as auditory or imaging data for example, can be utilized to supplement the bilateral asymmetry analysis, and thus can increase the specificity and accuracy of the system.

Traditional stroke assessments generally require expert examination, and are generally performed in a hospital or clinic setting. The presently disclosed technology can be utilized at nearly any location, at home, outdoors, at the office, and the like, including in various hospital and clinic settings. Subjects can be active to bedridden and still be continuously or intermittently monitored for potential stroke without the necessity of direct assessment by an expert health care provider. Upon the determination of a stroke or potential stroke condition, family, healthcare providers, emergency responders, or others, can be notified by the device so that swift medical attention can be provided. Such early detection of potential stroke conditions has the capacity to greatly reduce the time window between the onset of stroke symptoms and arrival at a medical facility, and thus facilitate more rapid access to medical treatment that can improve prognosis and reduce neurological damage.

Various potential stroke indicators can be monitored for signs of a potential stroke, either alone, or in combination, and any such indicator that can be diagnostic to a potential stroke assessment is considered to be within the present scope. Stroke indicators can generally include any physiological response, bodily sign, bodily movement, communication from the subject, or measurable phenomena that can be linked to a stroke or a potential stroke condition, including bilateral asymmetries. Non-limiting examples of potential stroke indicators can include bilateral asymmetries, bilateral motor asymmetries and motor asymmetries in general, facial asymmetries, speech pattern variations, pronator drift, drift, limb or body weakness, hemiplegia, atypical gait patterns, and the like, including combinations thereof.

One specific non-limiting example of a bilateral motor asymmetry that can be useful to monitor is referred to as drift, or pronator drift. In a neurological exam, a subject is tested for drift by being asked to hold both arms fully extended in front at shoulder level, and in some cases with the eyes closed. Drift is the inability for the subject to maintain this position. In general, an affected subject will experience a movement or drift of one arm relative to the other in either a downward or upward direction. In some cases, this drift can be accompanied by a pronation of the forearm, which can be referred to as pronator drift. It is additionally possible for a subject to experience pronation with little to no downward or upward drift. As used herein, pronator drift can be used to refer to such responses.

Thus by coupling sensors capable of measuring arm movement and/or rotation to each arm, and comparing the data output from each, movement and/or rotational asymmetries can be identified as suggestive of a potential stroke condition. Such asymmetries can then be analyzed further to assess the likelihood of an actual stroke condition. Additionally, the device can initiate further sensor data gathering and/or analysis of other data being gathered or previously gathered in order to facilitate the stroke condition assessment.

In practice, a sensor or sensors capable of detecting a potential stroke indicator such as a bilateral asymmetry are positioned such that relevant data can be recorded by the sensors from the subject. For example, accelerometers, gyroscopes, or both, can be positioned at correspondingly opposing locations of each hand, wrist, arm, etc., of the subject to measure drift and/or hemiplegia as potential stroke indicators. The data recorded from each sensor can be compared to identify any bilateral asymmetries that may be occurring. In this manner, a subject can go about their normal routine while being continuously or intermittently monitored for a potential stroke indicator.

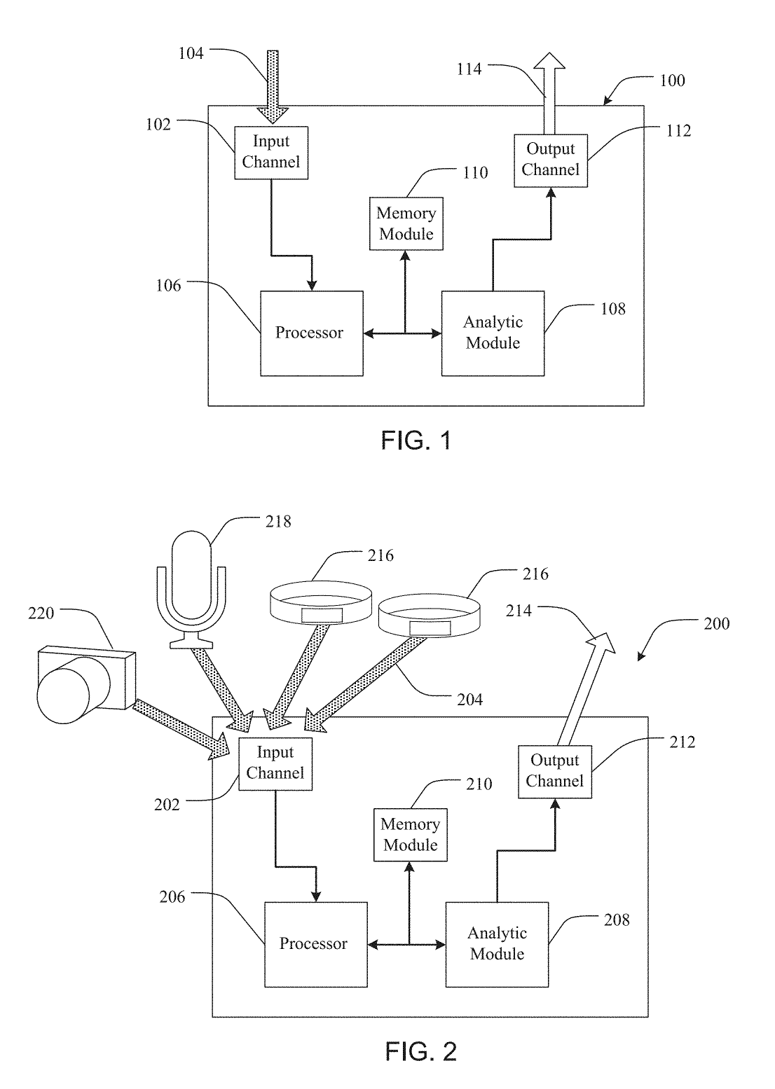

Turning to FIG. 1, for example, a device for analyzing sensor data to determine a potential stroke condition in a subject is shown. Such a device 100 can include an input channel 102 for receiving data 104 from a sensor or sensors associated with the subject, and a processor 106 coupled to the input channel 102. An analytic module 108 is shown coupled to the processor 106. The analytic module 108 can be a separate processor, an application or instruction set or portion thereof resident on or being executed by the processor 106, or any other analytic implementation of a data assessment algorithm. The analytic module 108 can identify, in conjunction with the processor 106, a potential stroke indicator in the data received from the input channel 102, and can further determine that the potential stroke indicator is suggestive of a stroke condition. A memory 110 can be coupled to the processor 106, and an output channel 112 for sending a notification 114 of the potential stroke condition can be coupled to the processor and/or the analytic module 108.

It should be noted that the arrangement shown in FIG. 1 is presented as an example, and it is understood that numerous designs can be implemented, each of which are considered to be within the present scope. In one general example, therefore, a device can include circuitry that is configured to: receive, using an input channel, data from a sensor or sensors associated with a subject, identify, using a processor, a potential stroke indicator in the data from the sensors, determine, using the processor, that the potential stroke indicator is suggestive of a stroke condition, and send, using an output channel, a notification of the potential stroke condition.

FIG. 2 shows an example of a system 200 for analyzing sensor data to determine a potential stroke condition in a subject. Such a system 200 can include a processor 206 and an input channel 202 coupled to the processor 206. An analytic module 208 is shown coupled to the processor 206. The analytic module 208 can be a separate processor, an application or instruction set or portion thereof resident on or being executed by the processor 206, or any other analytic implementation of a data assessment algorithm. The analytic module 208 can identify, in conjunction with the processor 206, a potential stroke indicator in data received from the input channel 202, and can further determine that the potential stroke indicator is suggestive of a stroke condition. A memory 210 can be coupled to the processor 206, and an output channel 212 for sending a notification 214 of the potential stroke condition can be coupled to the processor 206 and/or the analytic module 208. For this and other examples shown, the various connections are intended to show a limited representative pathway to preserve clarity. It is understood that numerous connections and interconnections are present that are not shown, but would be understood to be present to one of ordinary skill in the art, once in possession of the present disclosure. For example, in some examples the various elements of a device or system can be coupled through a local communication interface.

In one example system, instructions can be embodied on a non-transitory machine readable storage medium (i.e. memory 210) that when executed, perform the potential stroke indicator and potential stroke condition analysis described herein. Specifically, one example instruction when executed causes the system 200 to receive data 204 through the input channel 202 from a plurality of sensors 216, 218, 220 positioned to gather data from the subject, and to processes the data 204 using the processor 206 and/or the analytic module 208 to identify a potential stroke indicator. The processor 206 and/or the analytic module 208 then determine whether or not the potential stroke indicator is suggestive of or is a stroke condition, and send a notification 214 of the potential stroke condition to the output channel 212.

The input channels 102, 202 can vary depending on the design of the device or system, the mobility of the subject, the nature of the incoming data, and the like. The connection spanning from the sensor to the processor can be a physical connection, a wireless connection, a Bluetooth connection, an optical connection, a cellular connection, or the like, including combinations thereof. In one specific example, the input channel couples to the sensor via a wireless connection. In another specific example, the input channel couples to the sensor via a Bluetooth connection.

The input channels 112, 212 can vary depending on the design of the device or system, the mobility of the subject, the nature of the outgoing notification data, other device output considerations, and the like. In one example the output channel can send a notification that displays directly at the device or system, such as, for example, a light, sound, or other notification that can be detected by an individual in proximity to the device. In other examples, the output channel sends the notification to a remote device, either through a wired or a non-wired connection. Non-wired connections can include any connection capable of delivering the notification. Non-limiting examples include a wireless network connection, a Bluetooth connection, an optical connection, a cellular connection, The connection spanning from the sensor to the processor can be a physical connection, a wireless connection, a Bluetooth connection, an optical connection, or the like, including combinations thereof. In some cases a notification can be sent to multiple destinations, and therefore a combination of connection types can include a cellular call to one destination and a Bluetooth connection to another. Such would also include wireless and wired combinations, as well as visual or auditory notifications at the device. In one specific example, the output channel delivers the notification via a Bluetooth connection. In another specific example, the output channel delivers the notification via a cellular connection.

Depending on the potential stroke indicator being monitored, various sensor types can be utilized. Any sensor capable of detecting a potential stroke indicator is considered to be within the present scope. The example sensors shown in FIG. 2 include wristbands 216 that can include accelerometers, gyroscopes, or any other similar sensor devices, a microphone 218, and an imager device 220 such as a camera. In general, non-limiting examples of sensors can include accelerometers, magnetometers, gyroscopes, force gauges, pressure sensors, and any other type of sensor capable of measuring any potential stroke indication. Auditory data can be used to monitor for auditory stroke indicators such as slurred speech, while image data of the subject can be used to monitor for visual stroke indicators, such as facial asymmetries, drooping, abnormal body movements, and the like. Such auditory and/or image data can be utilized as a supplement to, or as a replacement for, the accelerometer and/or gyroscopic data. Regarding sensor data in general, it is noted that in some cases sensor data can be generated and processed in real time, while in other cases sensor data can be stored on a non-volatile memory storage device and subsequently analyzed after a delay in time.

In some cases, the sensors can be physically coupled to the subject. For example, monitoring motor asymmetries can be accomplished by physically coupling accelerometers to the subject. Thus by coupled an accelerometer to each hand or arm of the subject, the sensor outputs can be compared to identify a bilateral motor asymmetry such as drift or hemiplegia. The determination can be further facilitated by physically coupling a gyroscope to each and or arm of the subject due to the rotational sensitivity of such devices. It is additionally contemplated that the accelerometers can be replaced with gyroscopes.

In other cases, a sensor can be oriented toward but not physically coupled to the subject. Examples can include microphones, imagers, and the like. It is noted that physical separation from the subject is not required, and that in some cases a subject could be holding the sensor, and thus technically be physically coupled therewith. For example, an application on a smart phone may trigger an image to be periodically taken of the subject when the phone is in use. In this case, the smart phone containing the imager is being held by the subject, and thus is in physical contact therewith.

As described herein, a variety of sensor types can be utilized with the presently disclosed technology, and the data types that can be analyzed are similarly diverse. Non-limiting examples of data types can include accelerometer data, magnetometer data, gyroscopic data, acoustic data, image data, electrophysiological data, manometer data, and the like, including combinations thereof.

Accelerometer data can include numerous measurement metrics due to the broad utility of accelerometer sensors. For example, accelerometers are inertial sensors that can measure in one, two, or three orthogonal axes. Accelerometers can gather inertial measurements of velocity and position, as well as sense inclination, tilt, or orientation in two or three dimensions. These sensors can therefore be used to gather data relevant to drift and other motor and weakness stroke indicators by measuring arm velocity and position, orientation, inclination, tilt, etc. Thus, accelerometer data can include can include motor response data, orientation data, mechanomyogram data, gait data, and the like, including combinations thereof. Furthermore, gyroscope data can be used in conjunction with, or as a replacement for, accelerometer data. Gyroscope sensors detect orientation changes of the sensor, and can increase the accuracy of pronation measurements.

Electrophysiological data can include any type of data associated with biologically-derived electrical activity, including without limitation, electroencephalogram (EEG) data, electromyogram (EMG) data, electrocardiogram (ECG) data, electrooculogram (EOG) data, electrodermal (ED) data, and the like, including combinations thereof.

Acoustic data can include any type of data resulting from sound generated by the subject, or data involving the subject's interaction with sound. Non-limiting examples can include speech data, mechanomyogram data, acoustic or echo location data, and the like, including combinations thereof.

Image data can include any type of data captured with an imager device, such as without limitation, facial image data, body image data, ocular image data, three dimensional body position data, and the like, including combinations thereof.

The various system examples described herein can generally include a processor in communication with a memory, an input channel, and an output channel. As used herein, the term processor can include one or more general purpose processors, specialized processors such as VLSI, FPGAs, or other types of specialized processors. In one specific example, the processor can be an Integrated Sensor Hub (ISH) processor. ISH processors allow efficient and continuous data acquisition and analysis, with minimal power consumption.

Memory can include any device, combination of devices, circuitry, and the like that is capable of storing, accessing, organizing and/or retrieving data. Non-limiting examples include SANs (Storage Area Network), cloud storage networks, volatile or non-volatile RAM, phase change memory, optical media, hard-drive type media, and the like, including combinations thereof.

Systems can also include a local communication interface for connectivity between the various components of a given system. For example, the local communication interface can be a local data bus and/or any related address or control busses as may be desired.

Systems can also include an I/O (input/output) interface for controlling the I/O functions of the system, as well as for I/O connectivity to devices outside of the system. A network interface can also be included for network connectivity. The network interface can control network communications both within the system and outside of the system. The network interface can include a wired interface, a wireless interface, a Bluetooth interface, optical interface, and the like, including appropriate combinations thereof. Furthermore, a system can additionally include a user interface, a display device, as well as various other components that would be beneficial for such a system.

Various techniques, or certain aspects or portions thereof, can take the form of program code (i.e., instructions) embodied in tangible media, such as floppy diskettes, compact disc-read-only memory (CD-ROMs), hard drives, non-transitory computer readable storage medium, or any other machine-readable storage medium wherein, when the program code is loaded into and executed by a machine, such as a computer, the machine becomes an apparatus for practicing the various techniques. Circuitry can include hardware, firmware, program code, executable code, computer instructions, and/or software. A non-transitory computer readable storage medium can be a computer readable storage medium that does not include signal. In the case of program code execution on programmable computers, the computing device can include a processor, a storage medium readable by the processor (including volatile and non-volatile memory and/or storage elements), at least one input device, and at least one output device. The volatile and non-volatile memory and/or storage elements can be a random-access memory (RAM), erasable programmable read only memory (EPROM), flash drive, optical drive, magnetic hard drive, solid state drive, or other medium for storing electronic data. One or more programs that can implement or utilize the various techniques described herein can use an application programming interface (API), reusable controls, and the like. Such programs can be implemented in a high level procedural or object oriented programming language to communicate with a computer system. However, the program(s) can be implemented in assembly or machine language, if desired. In any case, the language can be a compiled or interpreted language, and combined with hardware implementations.

As has been described, a subject can be monitored with a plurality of sensors, the output of which is monitored for potential stroke indications. Once a potential stroke indication is identified, the system can perform further processing to determine the likelihood that the potential stroke indication is related to a stroke condition. Any form of processing that can lead to a determination of a stroke condition is considered to be within the present scope. In one example of further processing, the system can perform one or more steps of the FAST test, or any other useful stroke diagnostic test.

Various clinical tests have been shown previously to be reliable, and are widely used by neurologists for the assessment and diagnosis of stroke. One example is the Cincinnati test (CPSS), which has been recommended by many medical authorities worldwide. This test includes three physical findings: facial droop, arm drift (or pronator drift), and speech deficits. The guidelines to the general public regarding detection of stroke refer to a “FAST” self-examination that includes Face drooping, Arm weakness, and Speech difficulty (e.g. slurred speech). Thus in one example the system may have been triggered to perform further analysis once an arm drift stroke indicator was detected. Further processing following the FAST guidelines in such a case can include processing image data of the subjects face to detect facial drooping, acoustic data of the subjects speech to detect speech defects, or both. In addition, other potential stroke indicators can be processed that are generally not included in the FAST test to facilitate an accurate determination of a stroke condition.

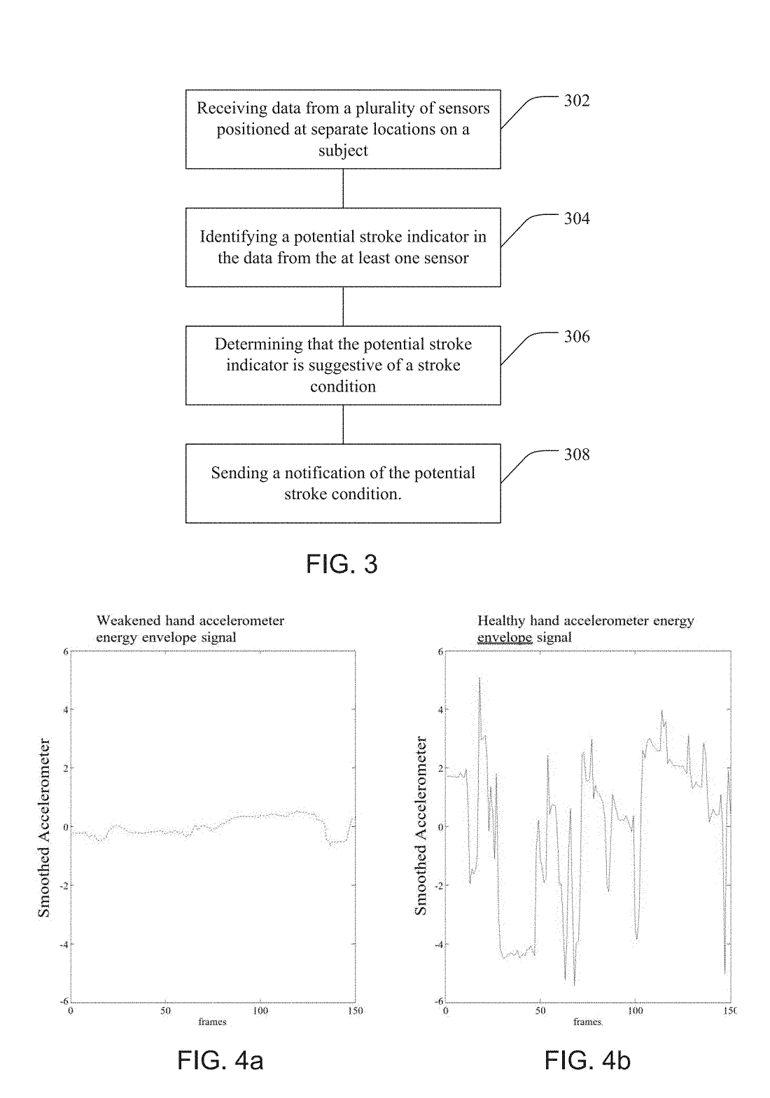

In one example, as is shown in FIG. 3, a method of determining a potential stroke condition in a subject is provided. Such a method can include 302 receiving, using an input channel, data from at least one sensor associated with a subject, 304 identifying, using a processor, a potential stroke indicator in the data from the at least one sensor, 306 determining, using the processor, that the potential stroke indicator is suggestive of a stroke condition, and 308 sending, using an output channel, a notification of the stroke condition. As such, the processor can continuously or intermittently process incoming sensor data to monitor for a potential stroke indicator or level of a potential stroke indicator above a specified threshold. In one example, the level of bilateral asymmetry can be assessed or otherwise determined in the data. A high level of bilateral asymmetry can indicate a potential stroke condition, and is therefore a potential stroke indicator. The level of bilateral asymmetry can be compared to a predefined threshold. Such can be useful as the threshold can be set according to an individual subject, thus correcting for bilateral asymmetries that may already be present.

The same or a different processor or application can then evaluate further the potential stroke indicator, including additional data, further details about the subject, and the like, to determine whether or not a stroke condition or potential stroke condition is occurring. This further evaluation can include any data processing, data integration, further data acquisition, or the like, that is capable of decreasing the likelihood of false positives. In some examples, the potential stroke indicator data can be reevaluated. In other examples, data from other sensors, either of the same or different sensor types, can be processed and evaluated in context with the potential stroke indicator. For example, if the potential stroke indicator is one of the FAST evaluation factors, sensor input from the other two evaluation factors can be integrated therewith to, in effect, perform an automatic FAST evaluation. Thus, upon detecting a potential stroke indicator of a bilateral arm weakness, speech data from an acoustic sensor and/or image data of the subjects face can be additionally evaluated for speech slurring and/or facial drooping. A similar evaluation can be performed if the potential stroke indicator is facial drooping or slurred speech.

In a further example, determining that the potential stroke indicator is suggestive of a stroke condition can include processing the sensor data to generate a response pattern, and matching the response pattern to a pre-generated stroke pattern. Such can be accomplished by a variety of methods, including various pattern recognition algorithms, machine learning algorithms, and the like.

In another example, a non-transitory machine readable storage medium is provided having instructions embodied thereon for analyzing sensor data to determine a potential stroke condition in a subject. When executed, the instructions cause the system to receive, using an input channel, data from at least one sensor associated with a subject, identify, using a processor, a potential stroke indicator in the data from the at least one sensor, determine, using the processor, that the potential stroke indicator is suggestive of a stroke condition, and sending, using an output channel, a notification of the stroke condition.

In some examples, sensor data can be preprocessed to facilitate further processing, regardless of the techniques utilized to evaluate the potential stroke condition. In one example, preprocessing the data includes filtering the data using a filter type selected from the group consisting of linear filters, non-linear filters, adaptive filters, and combinations thereof. Data can be filtered for a variety of reasons, non-limiting examples of which include reducing high frequency noise, removing outlier samples, smoothing the data, reducing false positive rates, and the like, including combinations thereof. In another example, preprocessing the data includes filtering the data with a linear filter. Non-limiting examples include Butterworth filters, Chebyshev filters, Elliptic low pass filters, window-based filters, frequency sampling-based filters, and the like, including combinations thereof. In one specific example, the preprocessing the data includes filtering the data with a Butterworth filter. In another example, processing the data includes filtering the data with an adaptive filter. Non-limiting examples of adaptive filters include least-mean squares filters, recursive least squares filters, and the like, including combination thereof. In another example, preprocessing the data includes filtering the data with a non-linear filter. Non-limiting examples of non-linear filters can include particle filters, Kalman filters, median filters, and the like, including combinations thereof.

The preprocessed data can further be segmented to facilitate further processing. It is noted, however, that in some examples data can be segmented and further processed without preprocessing. Segmentation can be accomplished by any known technique, non-limiting examples of which include energy envelope, zero crossing rate, Fourier transform, wavelet transform, and the like, including combinations thereof. In one specific example, segmentation of the data is by an energy envelope technique.

Following segmentation, indicator features can be extracted from the data that are related to the potential stroke indicator, and a feature pattern can be generated from these indicator features. Any technique capable of extracting indicator features from data is considered to be within the present scope. Non-limiting examples can include principle component analysis, linear predictive coding, wavelet coefficients, Fourier transforms, and the like, including combinations thereof. Where the data is acoustic data, examples of techniques for extracting indicator features can include mel-frequency cepstrum coefficients, cepstrum coefficients, delta mel-frequency cepstrum coefficients, delta cepstrum coefficients, linear predictive coding, parkor, and the like, including combinations thereof. For image data, examples of extraction techniques for indicator features can include gray level, discrete-cosine transform, textural features, and the like, including combinations thereof.

Various potential stroke indicators and indicator features can include any effect or characteristic useful in determining the likelihood of a stroke condition. For example, in data that includes accelerometer and gyroscopic data, rotation of the left and right hands of the subject can be a useful indicator feature. In other examples, an indicator feature can be any bilateral difference in any feature that is associated with a possible stroke condition. For example, features can be calculated for the left and right sides of a subject, and the differences in the feature vectors can be used as features for the bilateral asymmetry assessment. Non-limiting examples of potential stroke indicators in general can include bilateral asymmetries, motor asymmetries, facial asymmetries, speech pattern variations, pronator drift, drift, limb weaknesses, hemiplegia, atypical gait patterns, and the like, including combinations thereof.

The resulting feature pattern can be compared to a feature pattern generated by another sensor, or the feature pattern can be compared to a pre-generated pattern or model to arrive at a stroke condition determination. In one example, such a comparison can be made by using a technique such as machine learning, pattern recognition, or the like. Other non-limiting examples include linear predictive coding, hidden Markov models, neural networks, k-nearest neighbor, linear discriminate analysis, random forest, decision tree, adaptive boosting, Gaussian mixture models, dynamic time warping, lasso regressions, support vector machines, minimum distance classifiers, and combinations thereof. In another example, the comparison can be made by using a distance measure such as mean squared error, absolute error, correlation, coherence, and combinations thereof, to name a few. As described above, such any comparative technique can additionally be used to correlate the potential stroke indicator with a known stroke condition control.

The following description refers to sensors and stroke indications as accelerometers and gyroscopes detecting indicators of bilateral motor asymmetries and/or hemiparesis. It should be understood that this is for convenience only, and that other sensor types and/or stroke indications can be substituted where appropriate. In practice, individuals experiencing a stroke usually demonstrate significant motor asymmetry, and such asymmetry can be readily detected by the devices and systems described herein. Sensors, such as accelerometers and gyroscopes, are monitored either continuously or intermittently to detect potential stroke indications such as hemiparesis or other prominent indications. FIGS. 4a,b show examples of data of accelerometer signals recorded from each hand of a stroke-like subject. A stroke-like subject is defined as an individual that has not experienced a stroke, but is mimicking a stroke indication for the purposeless of experimental testing. In this case, the stroke-like subject was mimicking hemiparesis in the left hand (FIG. 4a), while the right hand (FIG. 4b) functioned as normal. A comparison of FIGS. 4a and 4b clearly shows the asymmetry in motor activity that is expected from a stroke condition.

One example method of detection of a stroke condition can be based on a machine learning algorithm. Such a method can be based on a representation of the sensor data in a “feature space,” i.e., using features to represent the data. The feature space can include various features that are aimed at representing the temporal dynamic of the data signals, including, for example, linear predictive coding coefficients, energy envelope, squared root mean squared, and the like. The sensor data can be data from one type of sensor, such as accelerometer data for example, or the sensor data can be from multiple sensor types, including image data, acoustic data, etc. Then, in order to represent the level of bilateral asymmetry for example, the difference between feature vectors is calculated and used as a final feature set.

In one example technique for generating the feature space, each sensor data signal is processed in several ways. First, the sensor data is preprocessed for various reasons, depending on the data type, the condition of the data, and the like. For example, sensor data can be filtered to reduce noise, or to enhance or otherwise emphasize relevant signal characteristics, waveforms, or features. Next, the sensor data is segmented, or in other words, divided into short segments (or blocks for images) such that each segment can be separately analyzed. Features are then extracted from the segmented data. Each segment (or block) of the signal is represented by features, which allows a more effective separation of data categories, such as healthy and stroke. In the present stroke assessment technology, bilateral asymmetry can be a beneficial stroke indicator. As such, it can be useful to calculate the differences between the left and right features as a measure of the level of bilateral asymmetry according to Equation I:

F₁=|Δf_{L}−Δf_{R}| I

where F₁ is the bilateral asymmetry feature, and Δf_{L} and Δf_{R} are acceleration measurements at the left sensor and the right sensor, respectively. These steps are performed on each signal, and the result is a matrix of features that represent the signal. This matrix is used in the training and testing processes.

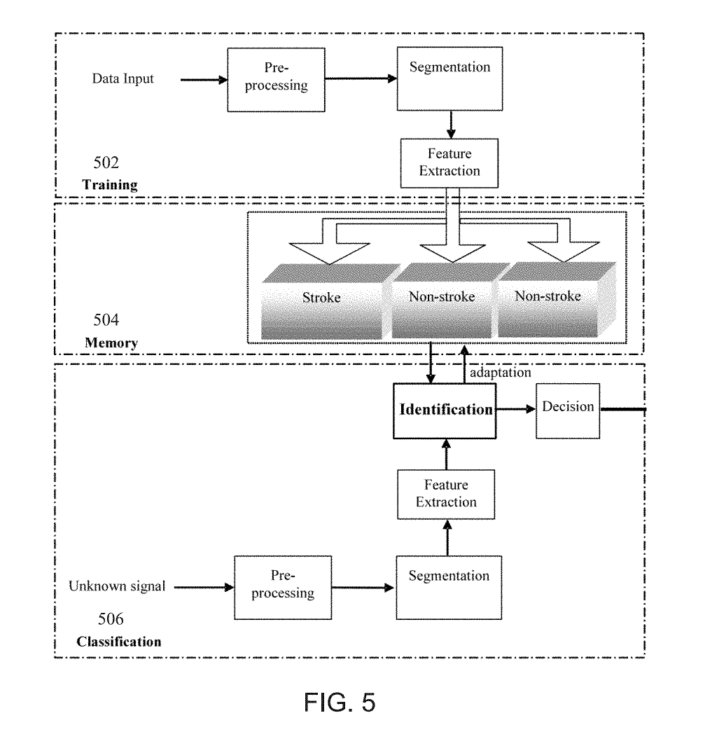

FIG. 5 shows an example of machine learning that can include a training phase 502, a memory phase 504, and a testing or classification phase 506. Note that for demonstration purposes, three models are shown in FIG. 5: two for stroke and one for non-stroke models. Since there can be a large variation between patients' symptoms, it can be useful to employ multiple models to reliably represent stroke. The training phase 502 can often be a one-time process, although training can be repeated for different sensors, different individuals, or alteration that may affect the stored models. Following generation, the patterns or models can be saved in memory.

During the training phase 502, the system is presented with examples of data from healthy and data from stroke patients, and based on this it estimates a parametric model or pattern for each category. The model is saved in memory and used during the testing phase. In one example, a Gaussian mixture model (GMM) can be used. Mixture models, and in particular GMM, form a common technique for probability density estimation. This is justified by the fact that any density can be estimated in a required degree of approximation, using finite Gaussian mixture. The mathematical properties of GMMs, as well as their flexibility and the availability of efficient estimation algorithms, make them useful for various classification problems. Training GMMs is performed using the expectation maximization (EM) algorithm, which is known in the art. This algorithm allows for iterative optimization of the mixture parameters, under monotonic likelihood requirements, and has a relatively simple implementation. According to the GMM concept, the positive and negative cases are represented by two classes, each of which is modeled by a GMM that is defined as a weighted sum of K Gaussian component densities and a useful tool for probability density function (pdf) representation. A GMM, representing a random process, x, can be expressed as per Equation II:

$${f}_{K} ( x ) = {\sum }_{k = 1}^{K} {π}_{k} {φ}_{{θ}_{k}} ( x ) \qquad II$$

where ϕ_{θ}_{ₖ} (x) represents the k th Gaussian mixture component and πₖ represents the mixing weight such that (Equation III):

$${\sum }_{k = 1}^{K} {π}_{k} = 1 , {π}_{j} \geq 0 ∀ j \qquad III$$

A multivariate Gaussian mixture is given by the weighted above-mentioned sum, where the jth component ϕ(x;θⱼ) is the d-dimensional Gaussian density (Equation IV):

ϕ(x;θⱼ)=(2π)^{-d/2}|sⱼ|^{-1/2}exp[−0.5(x−mⱼ)^{T}sⱼ⁻¹(x−mⱼ)] IV

which is parameterized on the mean mⱼ and the covariance matrix sⱼ, collectively denoted by the parameter vector θⱼ. During the training phase, the parameters of the GMMs are estimated using the EM algorithm, and saved in memory 504.

During the testing phase 506, or the regular usage of the system, the feature matrix of the incoming sensor data is generated (e.g., during the feature extraction phase), and the system calculates the probability or score that the particular testing matrix belongs to each one of the models that were saved in memory 504 during the training phase 502. The maximal score/probability determines which category the signal belongs to, in this case stroke or non-stroke. This probability/score calculation can be performed in various ways depending on the specific machine learning algorithm used.

For example, the classification criterion for GMM can be based on a maximum likelihood (ML) decision rule. According to this approach, the decision is made by finding the class, m, which maximizes the likelihood function (Equation V):

$$m = {argmax}_{m = 1 , 2 , … , M} {f}_{k} ( x ; {H}_{m} ) \qquad V$$

where fₖ(x;Hₘ) is the pdf of the classification features, x, under hypothesis Hₘ. These pdfs under the different hypothesis are estimated by the greedy GMM. In the present case, it can be useful to discriminate between two classes of cases, stroke and non-stroke. Therefore, a likelihood ratio test (LRT) can be a useful test, and can be employed as follows (Equation VI):

$$LRT = \frac{f ( x ; {H}_{2} )}{f ( x ; {H}_{1} )} \begin{matrix}> \\ <\end{matrix} γ \qquad VI$$

The log LRT (LLRT) is given by (Equation VII):

LLRT=In f(x;H₂)−ln f(x;H₁)_{<}^{>}γ′ VII

Furthermore, in some examples an adaptation algorithm can be used to adjust and optimize the testing model for a particular subject. This can beneficially reduce false positive rates in case of subjects who already suffer from motor asymmetry (e.g., due to a previous stroke). For example, the threshold γ′ may be updated (e.g., increased by a certain level) to optimize the test and reduce false positive rates. This can be performed, for example, based on feedback from the user in case of previous positive results. In another example, the parameters of the GMMs denoted as θⱼ, may be updated continuously using the EM algorithm, based on the new sensor data that were collected and the user's feedback.

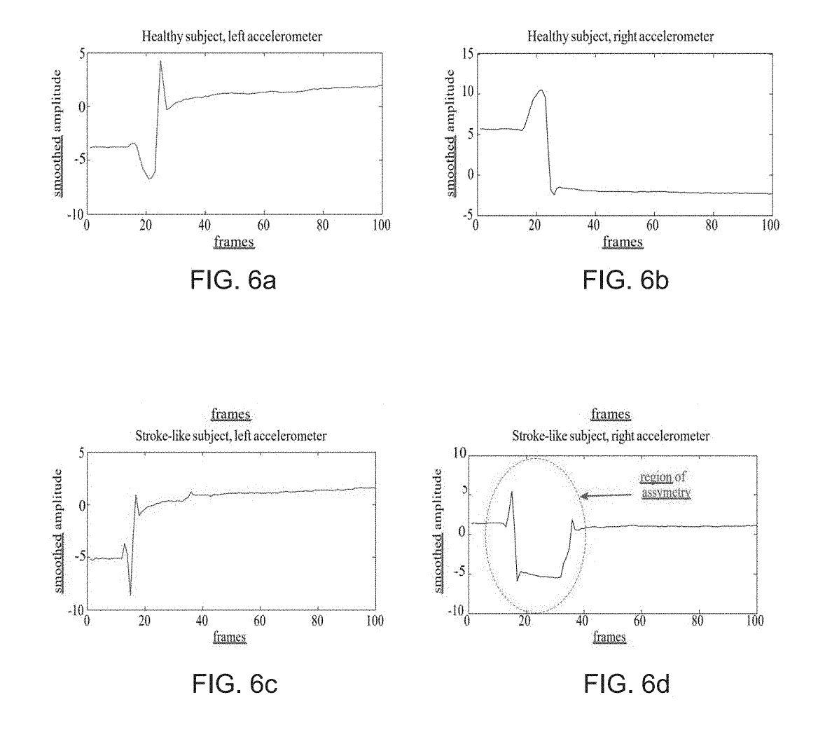

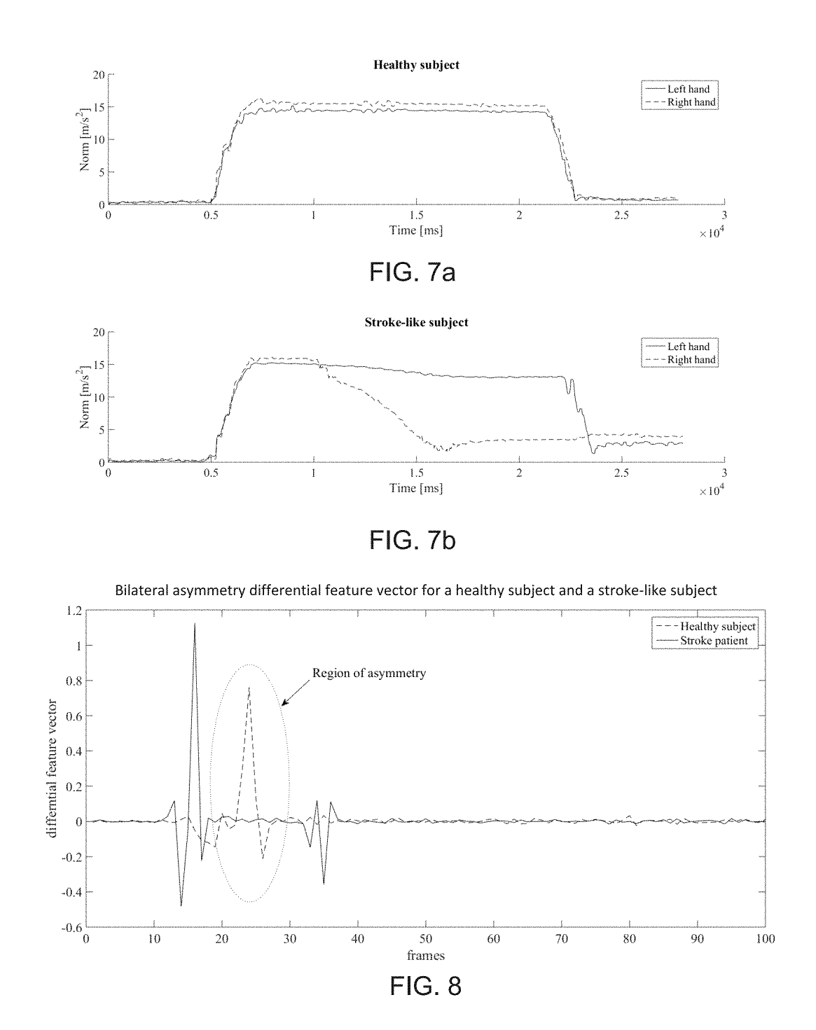

As an example, FIGS. 6a-d show bilateral accelerometer data from a healthy subject (FIGS. 6a-b) and a stroke-like subject (FIGS. 6a-c), and FIGS. 7a-b shows a comparison between the norm obtained from the healthy subject (FIG. 7a) and from the stroke-like subject (FIG. 7b). FIG. 8 shows the bilateral asymmetry differential feature vector calculated from the example. In all of these data, the differences between the two subjects can clearly be seen.

The following examples pertain to specific invention embodiments and point out specific features, elements, or steps that can be used or otherwise combined in achieving such embodiments.

In one example there is provided a device for analyzing sensor data to determine a potential stroke condition in a subject, the device comprising circuitry configured to:

receive, using an input channel, data from at least one sensor associated with a subject;

identify, using a processor, a potential stroke indicator in the data from the at least one sensor;

determine, using the processor, that the potential stroke indicator is suggestive of a stroke condition; and

send, using an output channel, a notification of the potential stroke condition.

In one embodiment of a device for analyzing sensor data to determine a potential stroke condition in a subject, the data from at least one sensor associated with the subject further comprises data from a plurality of sensors physically coupled to the subject at separate locations.

In one example of a device for analyzing sensor data to determine a potential stroke condition in a subject, at least one sensor of the plurality of sensors is oriented toward but not physically coupled to the subject.

In one example of a device for analyzing sensor data to determine a potential stroke condition in a subject, at least one sensor of the plurality of sensors is held by the subject.

In one example of a device for analyzing sensor data to determine a potential stroke condition in a subject, the at least one sensor is at least two sensors, and identifying the potential stroke indicator further comprises comparing the data from different sensors.

In one example of a device for analyzing sensor data to determine a potential stroke condition in a subject, in determining that the potential stroke indicator is suggestive of a stroke condition, the circuitry is further configured to:

process the data from the at least one sensor to generate a response pattern; and

match the response pattern to a pre-generated stroke pattern.

In one example of a device for analyzing sensor data to determine a potential stroke condition in a subject the data from the at least one sensor includes real-time sensor data.

In one example of a device for analyzing sensor data to determine a potential stroke condition in a subject the data includes accelerometer data.

In one example of a device for analyzing sensor data to determine a potential stroke condition in a subject the data further includes a data type selected from the group consisting of magnetometer data, gyroscopic data, acoustic data, image data, electrophysiological data, manometer data, and combinations thereof.

In one example of a device for analyzing sensor data to determine a potential stroke condition in a subject the data includes gyroscopic data.

In one example of a device for analyzing sensor data to determine a potential stroke condition in a subject the data includes a data type selected from the group consisting of accelerometer data, magnetometer data, gyroscopic data, acoustic data, image data, electrophysiological data, manometer data, and combinations thereof.

In one example of a device for analyzing sensor data to determine a potential stroke condition in a subject the electrophysiological data includes a member selected from the group consisting of electroencephalogram data, electromyogram data, electrocardiogram data, electrooculogram data, electrodermal data, and combinations thereof.

In one example of a device for analyzing sensor data to determine a potential stroke condition in a subject the accelerometer data includes a member selected from the group consisting of motor response data, orientation data, mechanomyogram data, gait data, and combinations thereof.

In one example of a device for analyzing sensor data to determine a potential stroke condition in a subject the acoustic data includes a member selected from the group consisting of speech data, mechanomyogram data, echo location data, and combinations thereof.

In one example of a device for analyzing sensor data to determine a potential stroke condition in a subject the image data includes a member selected from the group consisting of facial image data, body image data, ocular image data, three dimensional body position data, and combinations thereof.

In one example of a device for analyzing sensor data to determine a potential stroke condition in a subject the input channel includes a wireless receiver.

In one example of a device for analyzing sensor data to determine a potential stroke condition in a subject the output channel includes a wireless transmitter.

In one example of a device for analyzing sensor data to determine a potential stroke condition in a subject the processor includes an integrated sensor hub (ISH).

In one example of a device for analyzing sensor data to determine a potential stroke condition in a subject the potential stroke indicator includes an effect selected from the group consisting of a bilateral asymmetry, a motor asymmetry, a facial asymmetry, a speech pattern variation, pronator drift, drift, limb weakness, hemiplegia, atypical gait pattern, and combinations thereof.

In one example of a device for analyzing sensor data to determine a potential stroke condition in a subject the potential stroke indicator includes a bilateral asymmetry.

In one example of a device for analyzing sensor data to determine a potential stroke condition in a subject the potential stroke indicator further includes an effect selected from the group consisting of a motor asymmetry, a facial asymmetry, a speech pattern variation, pronator drift, drift, limb weakness, hemiplegia, atypical gait pattern, and combinations thereof.

In one example of a device for analyzing sensor data to determine a potential stroke condition in a subject the circuitry is further configured to preprocess the data.

In one example of a device for analyzing sensor data to determine a potential stroke condition in a subject preprocessing the data includes filtering the data using a filter type selected from the group consisting of linear filters, non-linear filters, adaptive filters, and combinations thereof.

In one example of a device for analyzing sensor data to determine a potential stroke condition in a subject preprocessing the data includes filtering the data to reduce high frequency noise, remove outlier samples, smooth the data, reduce false positive rates, or a combination thereof.

In one example of a device for analyzing sensor data to determine a potential stroke condition in a subject preprocessing the data includes filtering the data with a linear filter selected from the group consisting of Butterworth filters, Chebyshev filters, Elliptic low pass filters, window-based filters, frequency sampling-based filters, and combinations thereof.

In one example of a device for analyzing sensor data to determine a potential stroke condition in a subject preprocessing the data includes filtering the data with a Butterworth filter.

In one example of a device for analyzing sensor data to determine a potential stroke condition in a subject preprocessing the data includes filtering the data with an adaptive filter selected from the group consisting of least-mean squares filters, recursive least squares filters, and combination thereof.

In one example of a device for analyzing sensor data to determine a potential stroke condition in a subject preprocessing the data includes filtering the data with a non-linear filters selected from the group consisting of particle filters, Kalman filters, median filters, and combinations thereof.

In one example of a device for analyzing sensor data to determine a potential stroke condition in a subject the circuitry is further configured to segment the preprocessed data.

In one example of a device for analyzing sensor data to determine a potential stroke condition in a subject segmentation of the preprocessed data includes a technique selected from the group consisting of energy envelope, zero crossing rate, Fourier transform, wavelet transform, and combinations thereof.

In one example of a device for analyzing sensor data to determine a potential stroke condition in a subject segmentation of the data is by an energy envelope technique.

In one example of a device for analyzing sensor data to determine a potential stroke condition in a subject the circuitry is further configured to extract indicator features from the segmented data related to the potential stroke indicator.

In one example of a device for analyzing sensor data to determine a potential stroke condition in a subject the circuitry is configured to extract indicator features from the data using a technique selected from the group consisting of principle component analysis, linear predictive coding, wavelet coefficients, Fourier transforms, and combinations thereof.

In one example of a device for analyzing sensor data to determine a potential stroke condition in a subject the data includes accelerometer data and gyroscopic data, and the indicator features include rotation of left and right hands of the subject.

In one example of a device for analyzing sensor data to determine a potential stroke condition in a subject the indicator features includes bilateral differences in the data.

In one example of a device for analyzing sensor data to determine a potential stroke condition in a subject the data is acoustic data and the circuitry is configured to extract indicator features from the acoustic data using a technique selected from the group consisting of mel-frequency cepstrum coefficients, cepstrum coefficients, delta mel-frequency cepstrum coefficients, delta cepstrum coefficients, linear predictive coding, parkor, and combinations thereof.

In one example of a device for analyzing sensor data to determine a potential stroke condition in a subject the data is image data and the circuitry is configured to extract indicator features from the image data using a technique selected from the group consisting of gray level, discrete-cosine transform, textural features, and combinations thereof.

In one example of a device for analyzing sensor data to determine a potential stroke condition in a subject the circuitry is further configured to compare the data from the at least two sensors associated with the subject using a technique selected from the group consisting of machine learning, pattern recognition, and a combination thereof.

In one example of a device for analyzing sensor data to determine a potential stroke condition in a subject the circuitry is further configured to compare the data from the at least two sensors associated with the subject using a technique selected from the group consisting of linear predictive coding, hidden Markov models, neural networks, k-nearest neighbor, linear discriminate analysis, random forest, decision tree, adaptive boosting, Gaussian mixture models, dynamic time warping, lasso regressions, support vector machines, minimum distance classifiers, and combinations thereof.

In one example of a device for analyzing sensor data to determine a potential stroke condition in a subject the circuitry is further configured to compare the data from the at least two sensors associated with the subject using a distance measure selected from the group consisting of mean squared error, absolute error, correlation, coherence, and combinations thereof.

In one example of a device for analyzing sensor data to determine a potential stroke condition in a subject the circuitry is further configured to determine that the potential stroke indicator is suggestive of a stroke condition by correlating the potential stroke indicator with a known stroke condition control.

In one example of a device for analyzing sensor data to determine a potential stroke condition in a subject the circuitry is further configured to assess a level of bilateral asymmetry in the data.

In one example of a device for analyzing sensor data to determine a potential stroke condition in a subject the circuitry is further configured to compare the level of bilateral asymmetry to a predefined threshold.

In one example there is provided a system for analyzing sensor data to determine a potential stroke condition in a subject, comprising:

a processor;

an input channel coupled to the processor;

an output channel coupled to the processor; and

a non-transitory machine readable storage medium coupled to the processor and having instructions embodied thereon that when executed perform the following:
receiving, using the input channel, data from a plurality of sensors positioned to gather the data from a subject;
processing, using the processor, the data from the plurality of sensors to identify a potential stroke indicator;
determining, using the processor, that the potential stroke indicator is suggestive of a stroke condition; and
sending, using the output channel, a notification of the potential stroke condition.

In one example of a system for analyzing sensor data to determine a potential stroke condition in a subject, the system further comprises a plurality of sensors operable to be coupled at separate locations on a subject.

In one example of a system for analyzing sensor data to determine a potential stroke condition in a subject, at least two sensors of the plurality of sensors are accelerometer sensors.

In one example of a system for analyzing sensor data to determine a potential stroke condition in a subject, at least two sensors of the plurality of sensors are gyroscope sensors.

In one example of a system for analyzing sensor data to determine a potential stroke condition in a subject, at least two sensors of the plurality of sensors are magnetometer sensors.

In one example of a system for analyzing sensor data to determine a potential stroke condition in a subject, one of the plurality of sensors is an acoustic sensor or an image sensor.

In one example of a system for analyzing sensor data to determine a potential stroke condition in a subject, the plurality of sensors includes a sensor type selected from the group consisting of accelerometer sensors, magnetometer sensors, gyroscopic sensors, acoustic sensors, image sensors, electrophysiological sensors, manometer sensors, electrodermal sensors, and combinations thereof.

In one example of a system for analyzing sensor data to determine a potential stroke condition in a subject, the input channel includes a wireless receiver for receiving data from the plurality of sensors.

In one example of a system for analyzing sensor data to determine a potential stroke condition in a subject, the input channel includes a physical connection for receiving data from the plurality of sensors.

In one example of a system for analyzing sensor data to determine a potential stroke condition in a subject, the output channel includes a wireless transmitter for sending the notification of the potential stroke condition.

In one example of a system for analyzing sensor data to determine a potential stroke condition in a subject, the output channel includes a physical connection for sending the notification of the potential stroke condition.

In one example of a system for analyzing sensor data to determine a potential stroke condition in a subject, the processor includes an integrated sensor hub (ISH).

In one example there is provided a non-transitory machine readable storage medium having instructions embodied thereon for analyzing sensor data to determine a potential stroke condition in a subject, the instructions when executed perform the following:

receiving, using an input channel, data from a plurality of sensors positioned to gather the data from a subject;

comparing, using a processor, the data from the plurality of sensors to identify a physiological deficit between the separate locations;

determining, using the processor, that the physiological deficit is suggestive of a stroke condition; and

sending, using an output channel, a notification of the stroke condition.

In one example of a non-transitory machine readable storage medium having instructions embodied thereon for analyzing sensor data to determine a potential stroke condition in a subject, the data further includes a data type selected from the group consisting of accelerometer data, magnetometer data, gyroscopic data, acoustic data, image data, electrophysiological data, manometer data, and combinations thereof.

In one example of a non-transitory machine readable storage medium having instructions embodied thereon for analyzing sensor data to determine a potential stroke condition in a subject, the electrophysiological data includes a member selected from the group consisting of electroencephalogram data, electromyogram data, electrocardiogram data, electrooculogram data, electrodermal data, and combinations thereof.

In one example of a non-transitory machine readable storage medium having instructions embodied thereon for analyzing sensor data to determine a potential stroke condition in a subject, the accelerometer data includes a member selected from the group consisting of motor response data, orientation data, mechanomyogram data, gait data, and combinations thereof.

In one example of a non-transitory machine readable storage medium having instructions embodied thereon for analyzing sensor data to determine a potential stroke condition in a subject, the acoustic data includes a member selected from the group consisting of speech data, mechanomyogram data, echo location data, and combinations thereof

In one example of a non-transitory machine readable storage medium having instructions embodied thereon for analyzing sensor data to determine a potential stroke condition in a subject, the image data includes a member selected from the group consisting of facial image data, body image data, ocular image data, three dimensional body position data, and combinations thereof.

In one example of a non-transitory machine readable storage medium having instructions embodied thereon for analyzing sensor data to determine a potential stroke condition in a subject, the potential stroke indicator includes an effect selected from the group consisting of a bilateral asymmetry, a motor asymmetry, a facial asymmetry, a speech pattern variation, pronator drift, drift, limb weakness, hemiplegia, atypical gait pattern, and combinations thereof.

In one example of a non-transitory machine readable storage medium having instructions embodied thereon for analyzing sensor data to determine a potential stroke condition in a subject, the potential stroke indicator includes a bilateral asymmetry.

In one example of a non-transitory machine readable storage medium having instructions embodied thereon for analyzing sensor data to determine a potential stroke condition in a subject, the potential stroke indicator further includes an effect selected from the group consisting of a motor asymmetry, a facial asymmetry, a speech pattern variation, pronator drift, drift, limb weakness, hemiplegia, atypical gait pattern, and combinations thereof.

In one example of a non-transitory machine readable storage medium having instructions embodied thereon for analyzing sensor data to determine a potential stroke condition in a subject, the instructions when executed preprocess the data.

In one example of a non-transitory machine readable storage medium having instructions embodied thereon for analyzing sensor data to determine a potential stroke condition in a subject, preprocessing the data includes filtering the data using a filter type selected from the group consisting of linear filters, non-linear filters, adaptive filters, and combinations thereof.

In one example of a non-transitory machine readable storage medium having instructions embodied thereon for analyzing sensor data to determine a potential stroke condition in a subject, preprocessing the data includes filtering the data to reduce high frequency noise, remove outlier samples, smooth the data, reduce false positive rates, or a combination thereof.

In one example of a non-transitory machine readable storage medium having instructions embodied thereon for analyzing sensor data to determine a potential stroke condition in a subject, preprocessing the data includes filtering the data with a linear filter selected from the group consisting of Butterworth filters, Chebyshev filters, Elliptic low pass filters, window-based filters, frequency sampling-based filters, and combinations thereof.

In one example of a non-transitory machine readable storage medium having instructions embodied thereon for analyzing sensor data to determine a potential stroke condition in a subject, preprocessing the data includes filtering the data with a Butterworth filter.

In one example of a non-transitory machine readable storage medium having instructions embodied thereon for analyzing sensor data to determine a potential stroke condition in a subject, preprocessing the data includes filtering the data with an adaptive filter selected from the group consisting of least-mean squares filters, recursive least squares filters, or combination thereof.

In one example of a non-transitory machine readable storage medium having instructions embodied thereon for analyzing sensor data to determine a potential stroke condition in a subject, preprocessing the data includes filtering the data with a non-linear filters selected from the group consisting of particle filters, Kalman filters, median filters, and combinations thereof.

In one example of a non-transitory machine readable storage medium having instructions embodied thereon for analyzing sensor data to determine a potential stroke condition in a subject, the instructions when executed segments the preprocessed data.

In one example of a non-transitory machine readable storage medium having instructions embodied thereon for analyzing sensor data to determine a potential stroke condition in a subject, segmentation of the preprocessed data includes a technique selected from the group consisting of energy envelope, zero crossing rate, Fourier transform, wavelet transform, and combinations thereof.

In one example of a non-transitory machine readable storage medium having instructions embodied thereon for analyzing sensor data to determine a potential stroke condition in a subject, segmentation of the data is by an energy envelope technique.

The non-transitory machine readable storage medium of claim 72, further comprising In one example of a non-transitory machine readable storage medium having instructions embodied thereon for analyzing sensor data to determine a potential stroke condition in a subject, the instructions when executed extract indicator features from the segmented data related to the potential stroke indicator.

In one example of a non-transitory machine readable storage medium having instructions embodied thereon for analyzing sensor data to determine a potential stroke condition in a subject, the instructions when executed extract indicator features from the data using a technique selected from the group consisting of principle component analysis, linear predictive coding, wavelet coefficients, Fourier transforms, and combinations thereof.

In one example of a non-transitory machine readable storage medium having instructions embodied thereon for analyzing sensor data to determine a potential stroke condition in a subject, the data includes accelerometer data and gyroscopic data, and the indicator features include rotation of left and right hands of the subject.

In one example of a non-transitory machine readable storage medium having instructions embodied thereon for analyzing sensor data to determine a potential stroke condition in a subject, the indicator features includes bilateral differences in the data.

In one example of a non-transitory machine readable storage medium having instructions embodied thereon for analyzing sensor data to determine a potential stroke condition in a subject, the data is acoustic data and the circuitry is configured to extract indicator features from the acoustic data using a technique selected from the group consisting of mel-frequency cepstrum coefficients, cepstrum coefficients, delta mel-frequency cepstrum coefficients, delta cepstrum coefficients, linear predictive coding, parkor, and combinations thereof.

In one example of a non-transitory machine readable storage medium having instructions embodied thereon for analyzing sensor data to determine a potential stroke condition in a subject, the data is image data and the circuitry is configured to extract indicator features from the image data using a technique selected from the group consisting of gray level, discrete-cosine transform, textural features, and combinations thereof.

In one example of a non-transitory machine readable storage medium having instructions embodied thereon for analyzing sensor data to determine a potential stroke condition in a subject, the instructions when executed compare the data from at least two of the plurality of sensors associated with the subject using a technique selected from the group consisting of machine learning, pattern recognition, and a combination thereof.

In one example of a non-transitory machine readable storage medium having instructions embodied thereon for analyzing sensor data to determine a potential stroke condition in a subject, the instructions when executed compare the data from the at least two sensors associated with the subject using a technique selected from the group consisting of linear predictive coding, hidden Markov models, neural networks, k-nearest neighbor, linear discriminate analysis, random forest, decision tree, adaptive boosting, Gaussian mixture models, dynamic time warping, lasso regressions, support vector machines, minimum distance classifiers, and combinations thereof.

In one example of a non-transitory machine readable storage medium having instructions embodied thereon for analyzing sensor data to determine a potential stroke condition in a subject, the instructions when executed compare the data from the at least two sensors associated with the subject using a distance measure selected from the group consisting of mean squared error, absolute error, correlation, coherence, and combinations thereof.

In one example of a non-transitory machine readable storage medium having instructions embodied thereon for analyzing sensor data to determine a potential stroke condition in a subject, the instructions when executed determine that the potential stroke indicator is suggestive of a stroke condition by correlating the potential stroke condition with a known stroke condition control.

In one example of a non-transitory machine readable storage medium having instructions embodied thereon for analyzing sensor data to determine a potential stroke condition in a subject, the instructions when executed assess a level of bilateral asymmetry in the data.

In one example of a non-transitory machine readable storage medium having instructions embodied thereon for analyzing sensor data to determine a potential stroke condition in a subject, the instructions when executed compare the level of bilateral asymmetry to a predefined threshold.

In one example there is provided a method of determining a potential stroke condition in a subject, comprising:

receiving, using an input channel, data from a plurality of sensors positioned to gather the data from a subject;

comparing, using a processor, the data from the plurality of sensors to identify a physiological deficit between the separate locations;

determining, using the processor, that the physiological deficit is suggestive of a stroke condition; and

sending, using an output channel, a notification of the stroke condition.

In one example of a method of determining a potential stroke condition in a subject, the sensors comprise a plurality of sensors physically coupled to the subject at separate locations.

In one example of a method of determining a potential stroke condition in a subject, at least one sensor of the plurality of sensors is oriented toward but not physically coupled to the subject.

In one example of a method of determining a potential stroke condition in a subject, at least one sensor of the plurality of sensors is held by the subject.

1-90. (canceled)

91. A device for analyzing sensor data to determine a potential stroke condition in a subject, the device comprising circuitry configured to:
receive, using an input channel, data from at least one sensor associated with a subject;
identify, using a processor, a potential stroke indicator in the data from the at least one sensor;
determine, using the processor, that the potential stroke indicator is suggestive of a stroke condition; and
send, using an output channel, a notification of the potential stroke condition.

92. The device of claim 91, wherein the data from at least one sensor associated with the subject further comprises data from a plurality of sensors physically coupled to the subject at separate locations.

93. The device of claim 91, wherein the at least one sensor is at least two sensors, and identifying the potential stroke indicator further comprises comparing the data from different sensors.

94. The device of claim 91, wherein in determining that the potential stroke indicator is suggestive of a stroke condition, the circuitry is further configured to:
process the data from the at least one sensor to generate a response pattern; and
match the response pattern to a pre-generated stroke pattern.

95. The device of claim 91, wherein the data from the at least one sensor includes real-time sensor data.

96. The device of claim 91, wherein the data includes accelerometer data.

97. The device of claim 91, wherein the data includes a data type selected from the group consisting of accelerometer data, magnetometer data, gyroscopic data, acoustic data, image data, electrophysiological data, manometer data, and combinations thereof.

98. The device of claim 91, wherein the input channel includes a wireless receiver.

99. The device of claim 91, wherein the output channel includes a wireless transmitter.

100. The device of claim 91, wherein the processor includes an integrated sensor hub (ISH).

101. The device of claim 91, wherein the potential stroke indicator includes an effect selected from the group consisting of a bilateral asymmetry, a motor asymmetry, a facial asymmetry, a speech pattern variation, pronator drift, drift, limb weakness, hemiplegia, atypical gait pattern, and combinations thereof.

102. The device of claim 91, wherein the potential stroke indicator includes a bilateral asymmetry.

103. The device of claim 91, wherein the circuitry is further configured to preprocess the data.

104. The device of claim 91, wherein the circuitry is further configured to determine that the potential stroke indicator is suggestive of a stroke condition by correlating the potential stroke indicator with a known stroke condition control.

105. The device of claim 91, wherein the circuitry is further configured to assess a level of bilateral asymmetry in the data.

106. A system for analyzing sensor data to determine a potential stroke condition in a subject, comprising:
a processor;
an input channel coupled to the processor;
an output channel coupled to the processor; and
a non-transitory machine readable storage medium coupled to the processor and having instructions embodied thereon that when executed perform the following:
receiving, using the input channel, data from a plurality of sensors positioned to gather the data from a subject;
processing, using the processor, the data from the plurality of sensors to identify a potential stroke indicator;
determining, using the processor, that the potential stroke indicator is suggestive of a stroke condition; and
sending, using the output channel, a notification of the potential stroke condition.

107. The system of claim 106, further comprising a plurality of sensors operable to be coupled at separate locations on a subject.

108. A method of determining a potential stroke condition in a subject, comprising:
receiving, using an input channel, data from a plurality of sensors positioned to gather the data from a subject;
comparing, using a processor, the data from the plurality of sensors to identify a physiological deficit between the separate locations;
determining, using the processor, that the physiological deficit is suggestive of a stroke condition; and
sending, using an output channel, a notification of the stroke condition.

109. The method of claim 108, wherein the sensors comprise a plurality of sensors physically coupled to the subject at separate locations.

110. The method of claim 108, wherein at least one sensor of the plurality of sensors is oriented toward but not physically coupled to the subject.

111. The method of claim 108, wherein at least one sensor of the plurality of sensors is held by the subject.
